(19) United States
(12) Patent Application Publication
(10) Pub. No.: US 2004/0086879 A1
(43) Pub. Date: May 6, 2004

(54) Tripartite molecular beacons

(76) Inventors: Yingufu Li, Ontario (CA); Razvan Nutiu, Ontario (CA)

Correspondence Address: GOWLING, LAFLEUR HENDERSON LLP, SUITE 560, 120 KING STREET WEST, PO BOX 1045, LCD 1, HAMILTON, ON, L8N 3R4, CA

(21) Appl. No.: 10/322,605

(22) Filed: Dec. 19, 2002

Related U.S. Application Data
(60) Provisional application No. 60/341,234, filed on Dec. 20, 2001.

(51) Int. Cl. C12Q 1/68; C07H 21/04

(52) U.S. Cl. 435/6; 536/24.3

(57) ABSTRACT

Tripartite molecular beacons (TMBs), are disclosed that are readily adaptable to high throughput applications. Each tripartite molecular beacon comprises three oligonucleotide components. The first oligonucleotide forms a hairpin stem and loop structure and the second and third oligonucleotides each comprise a sequence complementary to opposite strands of the hairpin stem. The second oligonucleotide has a fluorophore attached thereto and the third oligonucleotide has a quencher attached thereto.

### FIELD OF THE INVENTION

The present invention is directed to a novel type of molecular beacon and uses therefor. More specifically, the present invention relates to tripartite molecular beacons (TMBs) that are particularly useful in high throughput screening.

### BACKGROUND OF THE INVENTION

Throughout this application, various references are cited in parentheses to describe more fully the state of the art to which this invention pertains. The disclosures of these references are hereby incorporated by reference into the present disclosure, and for convenience the references are listed in the appended list of references.

Nucleic acid probes are used to detect specific target sequences in a mixture. Hybridization of a nucleic acid probe to a complementary sequence is a highly specific event. Synthetic oligonucleotide probes can be made which are specific for any desired sequence.

Traditionally, hybridization assays detect target sequences that have been immobilized on a solid support using linear probes. Linear oligonucleotide probes, while useful, can be difficult to detect and there can be problems with background signals due to an excess of probe which may be non-specifically retained on the support. Unhybridized probes must be removed by extensive washing steps and this can be time consuming.

Some of the problems associated with fluorescent labeled linear probes were overcome by the development of molecular beacons (MBs). Molecular beacons are hairpin-shaped oligonucleotide probes that fluoresce only when they hybridize to their target. The hairpin shape of the molecular beacon causes mismatched probe/target hybrids to easily dissociate at a significantly lower temperature than exact complementary hybrids. This thermal instability of mismatched hybrids increases the specificity of molecular beacons, thus enabling them to distinguish targets that differ by a few or only a single nucleotide.

When conjugated with different fluorophores, molecular beacons can be used to differentiate different target sequences in the same sample.

Molecular beacons have several significant advantages over linear probes (Bonnet, et al., 1999; Bonnet et al., 1998). They work as simple fluorescent reporters for specific nucleic acid targets in hybridization assays without the need to separate the probe-target complex from excess probes. The signaling specificity is very high and similar nucleic acid targets that differ only in a single mismatch or deletion can be distinguished with precision. The fluorescence reporting is very sensitive and a fluorescence increase of up to two orders of magnitude can be observed when a matching target is introduced.

Molecular beacons (MBs) have been used in a variety of nucleic acid based detections.

For examples, molecular beacons were used to monitor the synthesis of specific nucleic acids in sealed reaction vessels (Tyagi et al., 1998; Leone, et al., 1998; Piateck, 1998; Vet et al., 1999), to perform one-tube assays to identify single-nucleotide variations in DNA (Kostrikis et al, 1998 a, 1998b; Giesendorf, 1998; Marras, 1999), and to detect specific RNA targets within living cells (Matsuo, 1998; Sokol et al., 1998).

Some potential uses of molecular beacons have been discussed in several patents including U.S. Pat. No. 5,925,517; U.S. Pat. No. 6,103,476 and U.S. Pat. No. 6,150,097.

Despite the aforementioned attributes, standard molecular beacons have some drawbacks. First of all, molecular beacons are expensive to make. Each molecular beacon has to be specially synthesized in order to covalently link the fluorophore and the quencher moieties onto a specific DNA probe. Each synthesized molecular beacon needs to be rigorously purified to remove any failed sequences. It is particularly important to eliminate probes which have a fluorophore attached but which lack the quencher because these molecules will cause high background fluorescence. Secondly, covalent integration of fluorophore and quencher with DNA offers no flexibility in fluorophore change. For the situations where two or more DNA probes with identical DNA sequences but with different fluorophores need to be used, multiple syntheses and purifications have to be carried out. Thirdly, for applications such as DNA microarrays that involve surface immobilization, molecular beacons either have to be deposited onto the surface or have to be synthesized directly on the surface. Since most fluorophores can be photo-bleached relatively easily, extreme care is needed during the immobilization process to prevent the photo bleaching of molecular beacons. Considering all of these limitations, the use of molecular beacons is not practical in various situations where it is desirable to detect hundreds or even thousands of different nucleic acid targets simultaneously or separately. For instance, it would be extremely expensive to construct a DNA chip that consists of hundreds or even thousands of different molecular beacons. Thus, there remains a real and unmet need for a novel format of molecular beacons that can solve the aforementioned problems associated with standard molecular beacons and make molecular beacons useful as affordable probes for high throughput applications.

### SUMMARY OF THE INVENTION

The present invention is directed to a novel type of molecular beacon called a tripartite beacon (TMB) which demonstrates numerous advantages over previously known molecular beacons. Unlike prior art beacons which require the covalent linkage of a fluorophore and a quenching moiety to each specific sequence, the beacon of the present invention utilizes a universal fluorophore containing DNA sequence and a universal quencher containing DNA sequence which are each capable of forming a duplex with a universal loop sequence.

Each tripartite molecular beacon comprises three oligonucleotide components. The first oligonucleotide forms a hairpin stem and loop structure and the second and third oligonucleotides each comprise a sequence complementary to opposite strands of the hairpin stem. The second oligonucleotide has a fluorophore attached thereto and the third oligonucleotide has a quencher attached thereto.

In one aspect of the invention, there is provided a tripartite probe comprising:

a) a first oligonucleotide having a first end segment, a second end segment and a probe segment intermediate the first and second end segments;

b) a second, fluorescent-labeled oligonucleotide (F-DNA) hybridized to a portion of the first end segment; and

c) a third, quencher-modified oligonucleotide (Q-DNA) hybridized to a portion of the second end segment, wherein the first end segment and the second end segment have complementary regions capable of forming the first oligonucleotide into a stem-loop structure.

In a preferred embodiment, the first end segment comprises a first oligonucleotide-binding segment and a first complementarity segment adjacent to the first oligonucleotide-binding segment, and the second end segment comprises a second complementarity segment complementary to the first complementarity segment and a second oligonucleotide-binding segment adjacent to the second complementarity segment and wherein the F-DNA hybridizes to said the oligonucleotide-binding segment and the Q-DNA hybridizes to the second oligonucleotide-binding segment.

The probe segment may comprise a known sequence complementary to a specific target sequence or it may contain a cloning site for insertion of any desired probe sequence.

In the absence of a target sequence, the first complementarity segment and the second complementarity segment hybridize to form a duplex, thereby bringing the F-DNA and the Q-DNA into proximity whereby fluorescence from the F-DNA is quenched by the Q-DNA. In the presence of a target sequence, the probe segment binds to the target sequence and forms a probe-target duplex, thereby spatially separating the F-DNA and the Q-DNA whereby fluorescence from the F-DNA can be detected. The melting point of the probe-target duplex is higher than the melting point of the stem formed between the complementarity regions.

In one embodiment, the fluorophore is covalently linked to one end of the second oligonucleotide and the third oligonucleotide has a quencher moiety attached at one end.

The invention also provides a kit for the detection of a target sequence. The kit comprises:

i)a loop oligonucleotide (L-DNA) comprising a probe sequence and complementary sequences on each side of said probe sequence;

ii) a fluorescent labeled oligonucleotide capable of hybridizing to said loop oligonucleotide on one side of said probe sequence; and

iii) a quencher modified oligonucleotide capable of hybridizing to the loop oligonucleotide on the other side of the probe sequence.

The probe sequence may comprise a sequence complementary to a target sequence or the probe sequence may comprise a restriction enzyme cloning site.

A method of preparing an array for detection of nucleic acid sequences is also provided.

The method comprises the steps of:

i)providing a loop oligonucleotide having a probe sequence and complementary end segments capable of forming a stem-loop structure;

ii)immobilizing said loop oligonucleotide on a surface;

iii)incubating said surface with a fluorophore labeled oligonucleotide complementary to a first region of said loop oligonucleotide and a quencher modified oligonucleotide complementary to a second region of said loop oligonucleotide wherein said fluorophore labeled oligonucleotide and said quencher modified oligonucleotide hybridize to said loop oligonucleotide and fluorescence is detected when said probe sequence binds to a complementary target sequence.

In a preferred embodiment the loop oligonucleotide is immobilized on the surface through free DNA ends. The loop oligonucleotide, the fluorophore labeled oligonucleotide and the quencher modified oligonucleotide can be combined prior to immobilization on the surface. Alternatively, the fluorophore labeled oligonucleotide and the quencher-modified oligonucleotide are added after the loop oligonucleotide is immobilized. They may be added sequentially.

In accordance with another aspect of the invention, there is provided a tripartite molecular beacon comprising:

i) a first oligonucleotide having a first arm segment, a body segment, and a second arm segment, said first and second arm segments having sufficient complementarity to one another to form an internal hairpin structure;

ii) a second oligonucleotide having a fluorescent reporter at one end, said second oligonucleotide comprising a sequence complementary to said first arm segment; and

iii) a third oligonucleotide having a quencher moiety at one end, said third oligonucleotide comprising a sequence complementary to said second arm segment.

The first and second arm segments anneal to form a first stem, the second oligonucleotide and the first arm segment form a second stem, and the third oligonucleotide and the second arm segment form a third stem.

Tripartite beacons in which the second oligonucleotide is complementary to the second arm segment and the third oligonucleotide is complementary to the first arm segment are also contemplated. In a preferred embodiment, the body portion of the first oligonucleotide includes a cloning site comprising multiple restriction enzyme sites.

In a further embodiment, a probe sequence complementary to a target sequence is cloned into the cloning site.

In yet another embodiment, the first oligonucleotide is synthesised including a probe sequence complementary to a target sequence.

Preferably, the probe sequence is intermediate to and adjoining said first and second arm segments and is capable of forming a double stranded hybrid with the target sequence, said double stranded hybrid having a first strength. The first and second arm have sufficient complementarity to each other to form, under predetermined detection conditions a double stranded stem hybrid having a second strength less than the first strength. Thus, in the presence of target, the stem hybrid will dissociate and allow the probe sequence to anneal to the target sequence. The second and third oligonucleotides form double stranded hybrid stems with the first arm segment and the second arm segment, respectively. These stems have a strength necessary to maintain the tripartite structure under the predetermined detection conditions.

According to another aspect of the invention there is provided a molecular beacon labeling kit comprising a first DNA sequence having a fluorophore attached at an end and a second DNA sequence having a quencher attached at an end, wherein said first DNA sequence and said second DNA sequence are complementary to opposite strands of a double stranded stem of DNA.

In accordance with another aspect of the invention, there is provided a method of preparing a tripartite molecular beacon, said method comprising:

i) preparing a loop sequence comprising a central sequence complementary to the sequence to be detected and 3′ and 5′ sequences partially complementary to each other, whereby said 3′ and 5′ sequences form a first stem at the region of complementarity; and

ii) interacting said loop sequence with a fluorophore labeled sequence and a quencher sequence wherein said fluorophore sequence is complementary to the 5′ end of said loop sequence and said quencher sequence is complementary to the 3′ end of said loop sequence and wherein said fluorophore sequence forms a second stem with said 5′ end of the loop sequence and the quencher sequence forms a third stem with the 5′ end of the loop.

In a further aspect of the invention, there is provided a tripartite molecular beacon comprising:

i) a fluorophore linked DNA sequence (F-DNA);

ii) a quencher linked DNA sequence (Q-DNA); and

iii) a loop DNA sequence (L-DNA) having a) a first segment complementary to said F-DNA, b) a second segment complementary to said Q-DNA, c) two short self-complementary sequences next to the F-DNA and the Q-DNA that are able to form an intramolecular stem and d) a probe sequence intermediate said two self-complementary sequences, wherein in the presence of a target sequence, said probe sequence forms a hybrid duplex with said target sequence thereby forcing the dissociation of said two self-complementary sequences.

In one embodiment, the F-DNA has a fluorophore covalently attached at the 5′ end and forms a duplex or stem with a segment at the 5′ end of the L-DNA and the Q-DNA has a quencher moiety at its 3′ end and forms a duplex or stem with a segment at the 3′ end of the L-DNA.

In another embodiment, the Q-DNA has a quencher at its 5′ end and forms a stem with the 5′segment of L-DNA and the F-DNA has a fluorophore at its 3′ end and forms a stem with the 3′ segment of L-DNA.

The present invention also provides for various uses of the tripartite molecular beacons.

The tripartite molecular beacons of the present invention can be used in a variety of ways. They are particularly useful for high throughput applications where the use of prior molecular beacons was prohibitively expensive. Furthermore, in light of their specificity and the flexibility of label, they can be used to differentiate between homozygotes and heterozygotes. To do this, one would simply attach two different dyes to the beacons complementary to the two alleles.

They can also be used for multiplexing. This technique refers to using several molecular beacons with different colored fluorophores to detect numerous targets in a single sample. For instance, they can be used to detect single nucleotide differences in a DNA sequence. The sequence to be tested is amplified with PCR in the presence of four molecular beacon probes, each differing only in the nucleotide in question (A, C, T, or G) and in the color of their fluorophores. The identity of the variant nucleotide is deduced by observing which of the molecular beacons fluoresces (i.e. binds to the PCR product).

### BRIEF DESCRIPTION OF THE DRAWINGS

Preferred embodiments of the invention are described below with reference to the drawings, wherein:

FIG. 1 illustrates the structure of a prior art molecular beacon;

FIG. 2 illustrates the structure of a tripartite molecular beacon of the present invention;

FIG. 3 illustrates duplex structures formed according to the present invention;

FIG. 4 is a comparison of the thermal denaturation profiles of a prior molecular beacon compared to a tripartite molecular beacon;

FIG. 5 is a further comparison of a prior molecular beacon with an exemplary tripartite molecular beacon;

FIG. 6 illustrates the profile obtained with TMBs having an additional periphery base pair;

FIG. 7 illustrates results obtained using standard F-DNAs and Q-DNAs and different L-DNAs;

FIG. 8 illustrates real-time detection using various tripartite molecular beacons; and

FIG. 9 illustrate the results from an exemplary array.

### DETAILED DESCRIPTION OF THE INVENTION

A typical molecular beacon (MB) is a synthetic oligonucleotide which is used to identify a specific target sequence. Molecular beacons of the prior art consist of four components; a loop, a stem, a 5′fluorophore and a 3′ quencher. The fluorophore (F) and can emit intensive fluorescence when it is excited, and the quencher (Q) is non-fluorescent but can engage in fluorescence resonance energy transfer (FRET) with the fluorophore to quench its fluorescence.

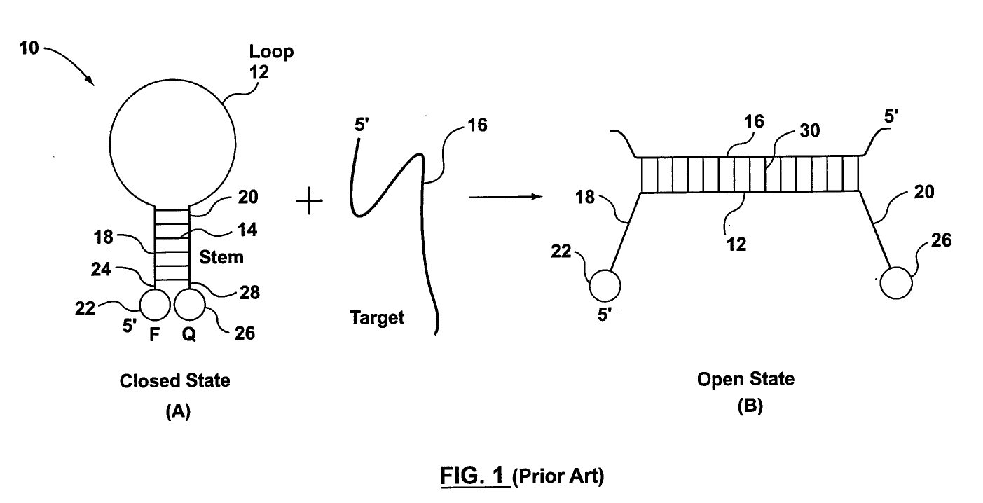

FIG. 1 illustrates schematically how a prior art molecular beacon works. The molecular beacon 10 has a loop 12 and a stem 14. The loop 12 includes a sequence complementary to a target nucleic acid sequence 16.

The stem 14 is formed by the annealing of complementary sequences 18, 20 at each end of the beacon. A fluorophore 22 is attached to the 5′ end 24 and a quenching moiety 26, also referred to as a quencher, is attached at the 3′ end 28. In the absence of a target nucleic acid sequence as shown in FIG. 1A, the molecular beacon forms an internal hairpin that brings the fluorophore 22 and the quencher 26 into close physical proximity. In this conformation, the fluorophore 22 is located within a short distance of the quencher 26 and therefore, the energy absorbed by the fluorophore is not emitted as fluorescence but is transferred to the quencher and the probe is not fluorescent. In the presence of a target nucleic acid sequence as shown in FIG. 1B, the molecular beacon 10 unfolds and the loop 12 anneals to the target nucleic acid sequence 16. This causes the fluorophore 22 and quencher 26 to become separated thereby enabling the detection of fluorescence emitted by the fluorophore. In other words, when a target nucleic acid sequence is introduced, a rigid helical structure 30, also referred to herein as a double stranded hybrid, is formed between the loop 12 of the molecular beacon and the target sequence 16 which forces the dissociation of the hairpin stem and the separation of the fluorophore from the quencher. Since a distantly located quencher is no longer able to absorb the energy from the excited fluorophore, the open state molecular beacon emits strong fluorescence. This occurs because the interaction between the target sequence and the probe sequence is stronger than the hybrid stem formed between the complementary sequences at the 3′ and 5′ ends of the beacon.

A major drawback to the prior art molecular beacons is that a unique beacon must be made for each target sequence. In each case, it is necessary to sequentially covalently link the fluorophore to one end and the quencher to the other end.

This novel molecular beacons of the present invention are called tripartite molecular beacons (TMBs). Similar to standard molecular beacons, a TMB has a significantly reduced fluorescence signal in its closed (i.e. hairpin) state due to high-efficiency fluorescence resonance energy transfer between the closely situated fluorophore and quencher.

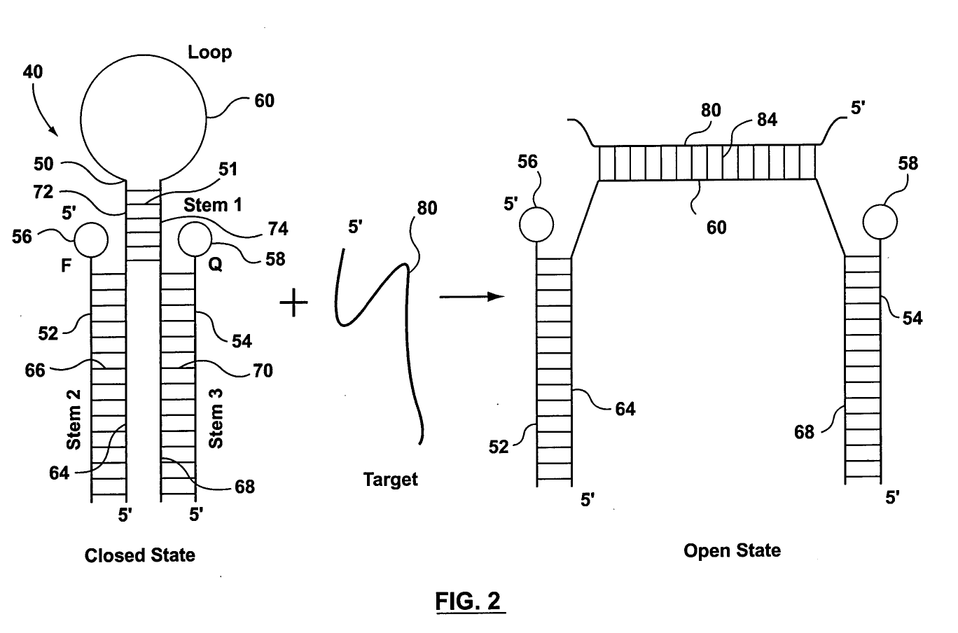

A tripartite molecular beacon 40 of the present invention is shown in FIG. 2. The tripartite beacon comprises three oligonucleotides, a first oligonucleotide 50 (denoted L-DNA) capable of forming a hairpin stem 51 similar to that seen with standard molecular beacons, a second oligonucleotide 52 (denoted F-DNA) and a third oligonucleotide 54 (denoted Q-DNA). The second and third oligonucleotides have sequences complementary to opposite strands of the hairpin stem 51. A fluorophore 56 is typically attached to the second oligonucleotide 52 (F-DNA) and a quencher 58 is typically attached to the third oligonucleotide 54 (Q-DNA).

It is also apparent that, rather than attaching a fluorophore or quencher to the F-DNA and Q-DNA respectively, the oligonucleotides can be synthesized using nucleotide analogs that have been modified to have flourescent or quencher properties. For example, fluorophore modified nucleotides are well known in the art. These include nucleotides where a fluorophore has been introduced into the ribose ring for example, other type of modified nucleotides are well-known to those skilled in the art.

In a preferred embodiment, the first oligonucleotide 50 or L-DNA is a standard, unmodified oligonucleotide that harbors a sequence 60 complementary to a target nucleic acid sequence. This complementary sequence is also referred to herein as a probe sequence. In practice, the L-DNA 50 typically comprises five sequence segments. The first segment 64 is the 5′ domain and is complementary to the F-DNA 52. The first segment 64 of the L-DNA and the F-DNA 52 together form an intermolecular stem 66, designated as Stem-2. The 3′ segment 68 is complementary to Q-DNA 54 and together they form another intermolecular stem 70, designated as Stem-3. Two short sequence motifs 72, 74, next to the F-DNA binding domain 64 and the Q-DNA binding domain 68 are self-complementary and form the intramolecular stem 51, designated as Stem-1. The segments of the L-DNA are also referred to herein as a first arm (comprising the 5′ segment 64 and complementary sequence 72), a body portion (comprising the probe sequence 60) and a second arm (comprising complementary sequence 74 and the 3′ segment 68).

It is clearly apparent that the positions of the F-DNA and the Q-DNA could be inverted. For example, the Q-DNA could have the quencher at its 5′ end and could form a stem with the 5′ segment of L-DNA and the F-DNA could have the fluorophore at its 3′ end and form a stem with the 3′ segment of L-DNA.

The probe sequence segment 60 is complementary to an external nucleic acid target 80. In the absence of a target sequence (FIG. 1A) the beacon is in a closed state due to the intramolecular stem 51.

The fluorophore 56 and the quencher 58 are in close proximity and the tripartite beacon does not fluoresce. In the presence of the target sequence 80, the intramolecular stem 51 dissociates, the tripartite beacon is converted to the open state and the probe sequence 60 and the target sequence 80 form a probe-target duplex 84. This separates the fluorophore 56 and the quencher 58 and fluorescence is emitted. This opening of the beacon occurs because the strength of the interaction between the two strands of stem 51 is less than the strength of the duplex 84 of the probe sequence 60 and the target sequence 80. In other words, fluorescence is very strong in the open state when a stronger probe-target duplex 84 is formed thereby forcing the dissociation of stem 51 and leading to the separation of the fluorophore 56 from the quencher 58.

Thus far, the description has focused on tripartite molecular beacons which include a probe sequence. It is, however, clearly apparent that the tripartite beacons of the present invention can also be provided as “empty” beacons into which one can insert any desired probe sequence. The body portion of the first oligonucleotide can include a cloning site comprising multiple restriction enzyme sites into which a desired probe sequence can be inserted.

The probe could also be provided as an “open-loop” probe in which each side of the so-called loop binds to a particular target. This type of TMB could be used to detect the simultaneous presence of two targets. For example, one-half of the “loop” could bind to an intron sequence and the other half of the “loop” could bind to an exon sequence. Generally, an “open-loop” TMB could be used to detect least two targets that are spatially separated. By virtue of the two halves of the loop binding to different sequences, the fluorophore and the quencher will be separated, thereby initiating a fluorescent signal. The detection of targets need not be limited to nucleic acid sequences. It is apparent that any target binding moiety, such as an antibody or a receptor, would be useful.

In addition, universal F-DNAs and Q-DNAs can be provided for the labeling of standard L-DNAs. For example, the universal F-DNA and Q-DNA can be interacted with a first oligonucleotide which is synthesised including a probe sequence complementary to a target sequence and F-DNA and Q-DNA binding domains.

Kits for the construction of tripartite molecular beacons are also included within the scope of the present invention.

For example, a kit can be provided which includes a first oligonucleotide with a multiple cloning site. Any desired probe sequence can be inserted into that site. The first oligonucleotide will contain regions of complementarity that result in a stem-loop structure in the absence of target. Universal F-DNA and Q-DNA can also be provided which hybridize with standardized sequences on the first oligonucleotide. Thus, one has only to insert the desired probe sequence into the multiple cloning site and then assemble the tripartitie molecular beacon. Of course, it is clearly apparent that kits comprising a first oligonucleotide with a particular probe sequence can also be provided. The TMBs of the present invention have the advantage over standard molecular beacons in that, since the fluorophore and quencher are not covalently linked to the ends of the probe sequence, there is the capability for surface immobilization through free DNA ends. The first oligonucleotide can be immobilized and the complementary F-DNA and Q-DNA can be added.

Methods for the production of tripartite beacons are also encompassed. A tripartite beacon is constructed by interacting three oligonucleotides having regions of complementarity as described above. This can be done in a variety of ways. F-DNA and Q-DNA can be pre-prepared having specific sequences. L-DNA can be prepared in a variety of ways, such as synthetically or recombinantly. The L-DNA must meet the criteria of i) sufficient complementarity to form an internal stem and ii) complementarity to the F-DNA and Q-DNA at opposite ends. Arrays or other solid surfaces can be coated with various L-DNAs which are then interacted with F-DNA and QDNA. The technology also allows for the development of solution phase assays.

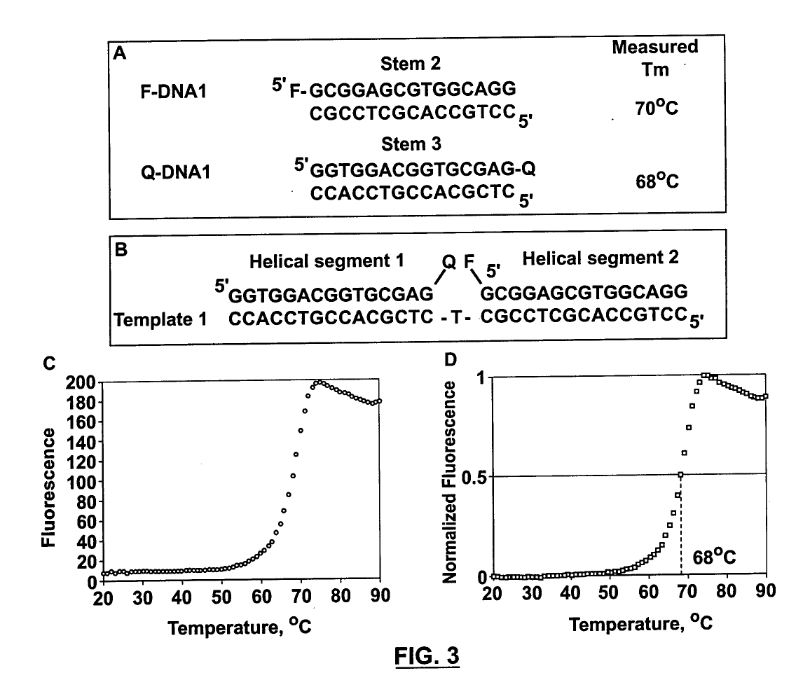

Referring now to FIG. 3, it is important that Stem 2 and Stem 3 are very stable so that the F-DNA and Q-DNA anneal strongly with L-DNA. This is particularly true for the temperature range of 20° C. to 50° C. within which nucleic acid hybridizations are usually carried out. The strong interaction between F-DNA and L-DNA as well as between Q-DNA and L-DNA can be achieved by having a high GC content in both stems. This is illustrated in FIG. 3A. Stem 2 contains 12 GC pairs out of 15 base-pairs and has an observed Tm of 70° C., determined from the thermal denaturation using absorption spectroscopy with a solution containing 10 mM Tris●HCI (pH8.3 at 23° C.), 0.5M NaCl, 3.5 mM MgCl2 and 0.1 ˜M of DNA. Stem 3 contains 11 GC pairs in 15-by duplex and has a similar melting point (Tm=68° C.) determined using the same method and under the same set of conditions. The formation of stable duplexes in Stem 2 and Stem 3 strongly links F-DNA and Q-DNA with L-DNA.

The experiment was repeated with the results that stem 2 (duplex 1) had a melting point of 68° C. and stem 3 (duplex 2) had a melting point of 66° C. These results confirm that the duplexes formed are very stable, most likely due to the high GC content.

Referring now to FIG. 3B, the ability of Q-DNA to quench the fluorescence of F-DNA was tested. A linear duplex was used in which Template 1 forms a duplex structure with F-DNA1, having fluorescein attached at its 5′ end and with Q-DNA1, having DABCYL linked to its 3′ end. The duplex structure contains two helical segments separated by a single unpaired nucleotide. The formation of the two helical structure elements will bring the fluorophore and the quencher into close proximity. Therefore, the fluorescence of F-DNA1 should be quenched when the three DNA oligonucleotides are mixed under nondenaturing conditions.

FIG. 3C shows the change of the fluorescence intensity as the temperature of the DNA mixture was increased. As expected, the fluorescence of the mixture was very low at low temperature range when FDNA1 and Q-DNA1 were fully assembled onto Template 1. At high temperatures when F-DNA1 and Q-DNA1 were dissociated from the template, strong fluorescence was observed. This indicates that closely located Q-DNA1 can indeed efficiently quench the fluorescence by F-DNA1. A plot of the normalized fluorescence, calculated as the ratio of the net fluorescence intensity (i.e., the fluorescence intensity at a particular temperature deducted by the intensity at 20° C.)emitted by the solution at a particular temperature over the maximal net intensity, is illustrated in FIG. 3D. From the plot, an apparent Tm is calculated to be 68° C., which matches the Tm of Stem 2. It is interesting to note that the maximal fluorescence of the mixture was found to be at 74° C. When the temperature is further increased above 74° C., the fluorescence intensity starts to decrease slowly. Examination of the solution containing only F-DNA1 revealed that F-DNA1 has fluorescence decreasing linearly with the increase of temperature (data not shown). This suggests that F-DNA1 and Q-DNA1 are completely separated at 74° C. and the further decrease in fluorescence beyond 74° C. simply reflects the intrinsic temperature dependence of F-DNA1.

From both FIGS. 3C and 3D, it is apparent that the properly annealed F-DNA1 and Q-DNA1 have stable and low fluorescence within the temperature range of 20° C. to 50° C. For example, the fluorescence intensity at 37° C. was only about 10% higher than that at 20° C. Even at 50° C., the solution had a fluorescence that was only about 40% higher than that at 20° C. The data indicates that F-DNA1 and Q-DNA1 can form duplex structures that are stable in the temperature range used in most nucleic acid hybridization experiments. Therefore, the tripartite molecular beacons should behave similarly to the standard unimolecular beacons with covalently attached F and Q.

To test this hypothesis, a tripartite molecular beacon, TMB1, was made and compared to a closely related standard molecular beacon, MB1. TMB1 comprises F-DNA1, Q-DNA1, and L-DNA1. L-DNA-1 has a sequence of 5′ CCTGCCACGCTCCGC GCGAGCCACCAAATATGATATGCTCGC-CTCGCACCGTCCACC-3′. The F-DNA1 binding sequence is shown in bold, the Q-DNA1 binding domain is indicated in italic and the self-complementary motifs are underlined. MB1 has the sequence of 5′-F-GCGAGCCACCAAATATGATATGCTCGC-Q-3′ (F: Fluorescein; Q: DABCYLTM). Therefore, TMB1 and MB1 share identical internal stem and loop 5 sequences.

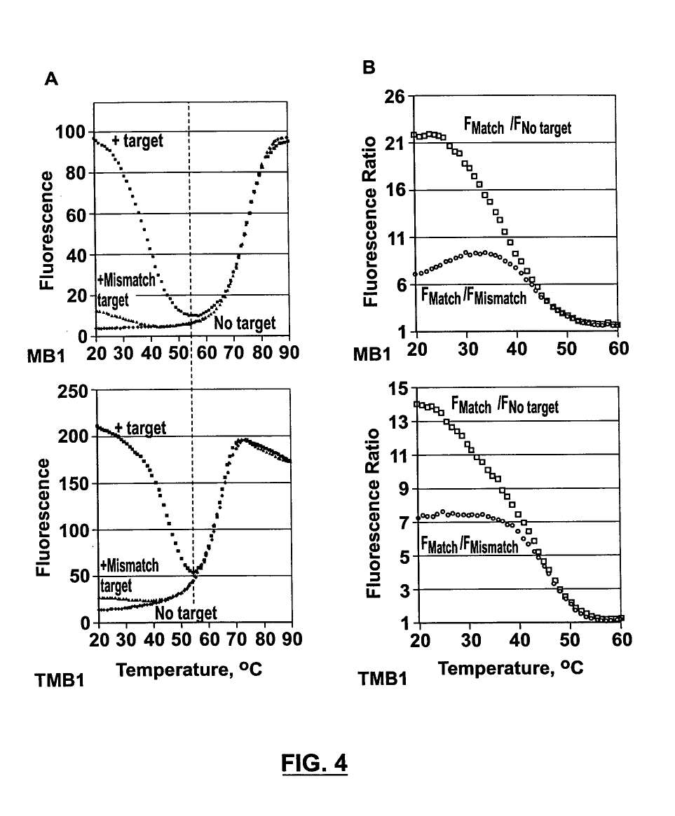

Various thermal denaturation profiles were obtained for both TMB1 and MB1 by heating relevant DNA mixtures (in 10 mM Tris-HCl, pH8.3, 0.5M NaCl and 3.5 mM MgCl2) to 90° C. to fully denature DNA structures and then cooling the mixtures to 20° C. in a controlled speed (2° C./min) to let the DNA molecules anneal. Fluorescence intensities were collected every 0.5° C. and are plotted in FIG. 4A. Both MB1 (top) and TMB1 (bottom) were examined under three sets of conditions: in the absence of a target (diamonds) with a matched target (squares) and in the presence of a mismatch target (triangles).

The overall behaviors of MB1 and TMB1 were similar under each condition, particularly within the lower temperature range (from 20° C. to 55° C.). In the absence of a nucleic acid target, the fluorescence in both systems experienced a rapid drop when the fully denatured solution was cooled to pass the point at which the intramolecular stem started to form to bring F and Q into close proximity. The intensity was stabilized at approximately 60° C. and below for MB1 and at below approximately 50° C. for TMB1 when most of the molecules are in the closed structure state. When the match target was used, the fluorescence intensity in both systems reached a minimal value at approximately 54° C. (dashed line in FIG. 4A). Further decrease in temperature resulted in rapid fluorescence increase due to the formation of rigid duplex structures between the DNA target and the loop sequence and the dissociation of the internal stem. The fluorescence increase was highly specific since another target that contained a single mismatch caused only small fluorescence increase in both systems.

There are visible differences in the two systems. Firstly, compared to MB1, TMB1 had the fluorescence intensity about twice as high. Since DNA concentrations were determined spectroscopically and the contribution to the absorbance at 260 nm by covalently attached fluorescein and/or DABCYL was not taken into the consideration, the intensity difference might simply reflect this inaccuracy. Secondly, MB1 had an apparent Tm of 74.5° C. that was 12° C. higher than that of TMB1 ( 62.5° C.). The observed melting point of MB1 is in excellent agreement with the calculated Tm of 74.2° C. (in 1 M NaCl) by M-fold program (http:/bioinfo.math.rpi.eduhmfold/dna/form1.cai). The smaller Tm observed for TMB1 is likely due to the following two reasons: (1) Stem 2 and Stem 3 have an observed Tm of 68° C. in the linear duplex described above, therefore it is not possible for TMB1 system to have an observed Tm above 68° C.; and (2) the base-pair at the outside edge of Stem 1 in TMB1 is very likely not able to form due to the severe congestion at the location where Stem 1, Stem 2 and Stem 3 meet. With the assumption that this base-pair is not formed, the M-fold program predicts a Tm of 63.0° C. for TMB1 (in 1 M NaCl), which matches quite well with the observed melting point of 62.5° C. Several other TMBs have also been examined for melting points and the observed Tm values were consistent with the assumption that the outside edge base-pairs are not formed. Thirdly, TMB1 had a unique appearance at high temperature in that the fluorescence intensity increased when temperature was dropped from 90° C. to 74° C. Similar behavior was observed in the linear duplex. We speculate that at 74° C. the tripartite system was completely denatured and the fluorescence increase with reduced temperature may simply reflect the intrinsic temperature dependence of single-stranded F-DNA1.

FIG. 4B is a plot of the fluorescence ratios vs. temperature for both MB1 and TMB1. Two kinds of ratios are given: the ratio of fluorescence intensity in the presence of the match target over the intensity in the absence of any target (i.e., FMatch/FNotarget˜open squares) and the ratio of the fluorescence intensity with the match target and with the mismatch target (i.e., FMatch/FMismatch˜open circles). FMatch/FNotarget measures the fluorescence enhancement when a target is introduced. For MB1, a maximum of −22-fold signal enhancement was observed between 20° C. and 25° C. The signal-to-background ratio decreased almost in a linear rate of 1-fold/degree between 29° C. and 43° C. For TMB1, the maximal fluorescence enhancement when the target was introduced was smaller at 14 fold and holds fairly steady between 20° C. and 25° C. The signal-to-background ratio decreases in a slower pace with a near linear rate of −0.5 fold/degree between 29° C. and 47° C. Although MB1 clearly has a better S/N ratio than TMB1, the difference is not very substantial.

FMatch/FMismatch measures the capability of an MB or a TMB to discriminate a perfect match target and a target with a single point mutation.

MB1 holds a slight edge again over TMB1 as MB1 produces a maximal 9.5-fold discrimination while TMB1 has a maximum of 7.5 fold. However, TMB1 has almost an unchanged discrimination ability within the temperature range of 20° C. to 37° C. MB1 on the other hand, has a reduced discrimination capability at 20° C. (7 fold) while maximizing out at 32° C. (9.5 fold). Nevertheless, MB1 and TMB1 have very comparable capability for single nucleotide discrimination.

F-DNA, Q-DNA and L-DNA are assembled through simple WatsonCrick hydrogen-bonding interactions into tripartite molecular beacon systems. Since F-DNA and Q-DNA are not directly involved in target binding, they can be universally used to construct any molecular beacon with a standard oligonucleotide (L-DNA) as long as F-DNA and Q-DNA do not affect the formation of the intended hairpin structure by L-DNA. This is a significant advantage over the prior molecular beacons since the three components can be simply combined. This makes TMBs much more practical and cost-effective than MBs for high throughput applications since there is no need to covalently modify every probe with the fluorophore and quencher pair.

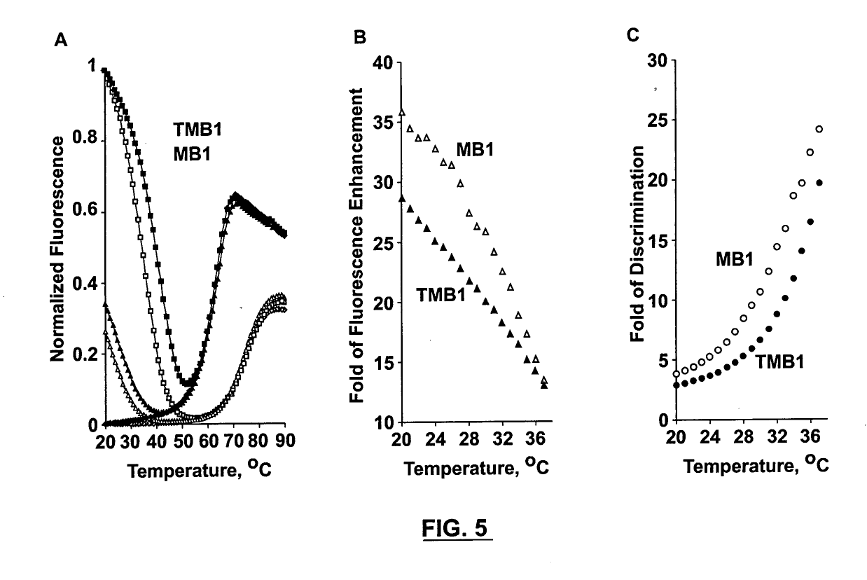

FIG. 5 is further comparison of TMB1 and MB1. Referring to FIG. 5A, fluorescence intensity was measured as a function of temperature in the absence of a nucleic acid target for MB1 (unfilled diamonds) and for TMB1 (filled diamonds). In the presence of the match target d(TACTCTTATATCATATTTGGTGTTTGCTTT)] for MB1 (unfilled squares) and for TMB1 (filled squares), as well as in the presence of a single-mismatch target [d(TACTCTTATATCATcTTTGGTGTTTGCTTT), the small letter indicates the single base mutation relative to the match target] for MB1 (unfilled triangles) and for TMB1 (filled triangles). TMB1 is made of FDNA1, QDNA1 (sequences shown in FIG. 1C), and LDNA1 with a sequence of d(CCTGCCACGCTCCGCGCGAGCCACCAAATATGATATGCTCGCCTCGCACCGTCCACC)(FDNA1-binding sequence shown in bold, QDNA1 binding domain indicated in italic and self-complementary motifs underlined). MB1 has the sequence of F-d(GCGAGCCACCAAATATGATATGCTCGC)-Q(F: Fluorescein; Q: DABCYL). The fluorescence intensity was normalized for each system as [(F_{T° C.})−(F_{20° C., no-target.}]/[(F_{20° C., match})−(F_{20° C., no-target}).] where (F_{T° .C}) is the fluorescence reading of a solution at any designated temperature, (F_{20° C., no-target}) and (F_{20° C., match}) are the readings at 20° C. for the samples containing no target and the match target, respectively. Referring now to FIG. 5B, the signal-to-background fluorescence ratio, calculated as (FT_{T° C., match})/(F_{T° C., no-target}), is plotted for MB1 (open triangles) and for TMB1 (filled triangles). FIG. 5C illustrates the single nucleotide discrimination capability. This is calculated as (F_{T° C. match}−F_{T° C. no-target})/(F_{T° C., mismatch}−F_{TC, no-target}) and is plotted for MB1 (open circles) and TMB1 (filled circles). (F_{T° C., match}),(F_{T° C., no-target}) and (F_{T° C., mismatch}) are the fluorescence readings for the samples containing match target, no target and mismatch target all at same temperature.

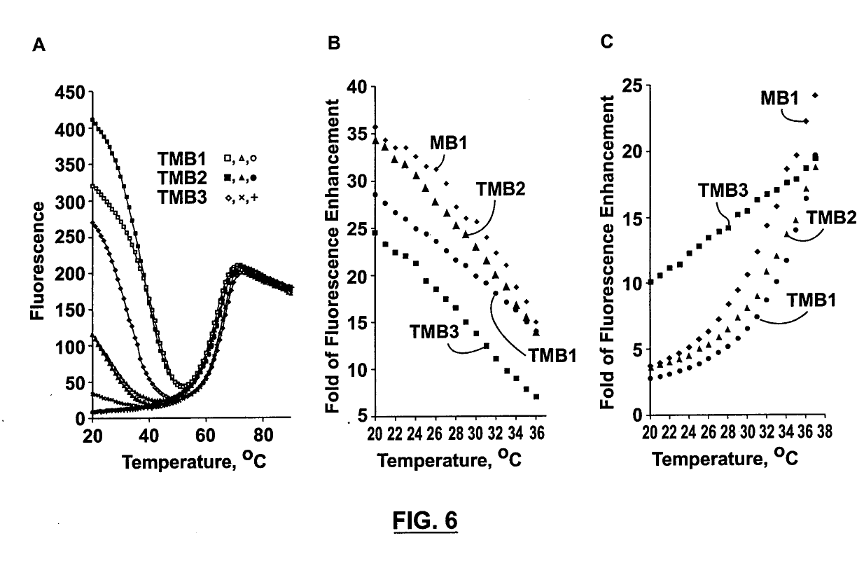

FIG. 6 illustrates the results obtained with TMBs having an additional periphery base pair. In FIG. 6A, both MB2[d(CCTGCCACGCTCCGCaGCGAGCCACCAAAT ATGATATGCTCGCtCTCGCACCGTCCACC)] and TMB3[d(CCTGCCACGCTCCGCgGCGAGCCACCAAATATGATATGC TCGCcCTCGCACCGTCCACC)] have the same sequence as TMB1 except for the base insertions (shown in small letters; FDNA1 binding sequence shown in bold, QDNA1 binding domain indicated in italic and self-complementary motifs underlined). Fluorescence intensity was measured as a function of temperature in the absence of nucleic acid target (diamonds), in the presence of the match target (squares) and as well as in the presence of a mismatch target (triangles). Match and mismatch target nucleic acid sequences are given in FIG. 5. FIG. 6B illustrates the signal-to-background fluorescence ratio, and FIG. 6C illustrates the single nucleotide discrimination capability for TMB2 (filled triangles), TMB3 (filled squares), as well as for MB1 (filled diamonds) and TMB1 (filled circles).

Since FDNA and QDNA are not directly involved in target binding, they can be used as a universal fluorophore/quencher pair to construct any molecular beacon with a standard DNA oligonucleotide (LDNA) as long as FDNA and QDNA do not affect the formation of the intended hairpin structure of LDNA. This makes TMBs an alternative and cost-effective form of molecular beacon for applications that require large number of probes, since there is no need to covalently modify every probe with a fluorophore and a quencher.

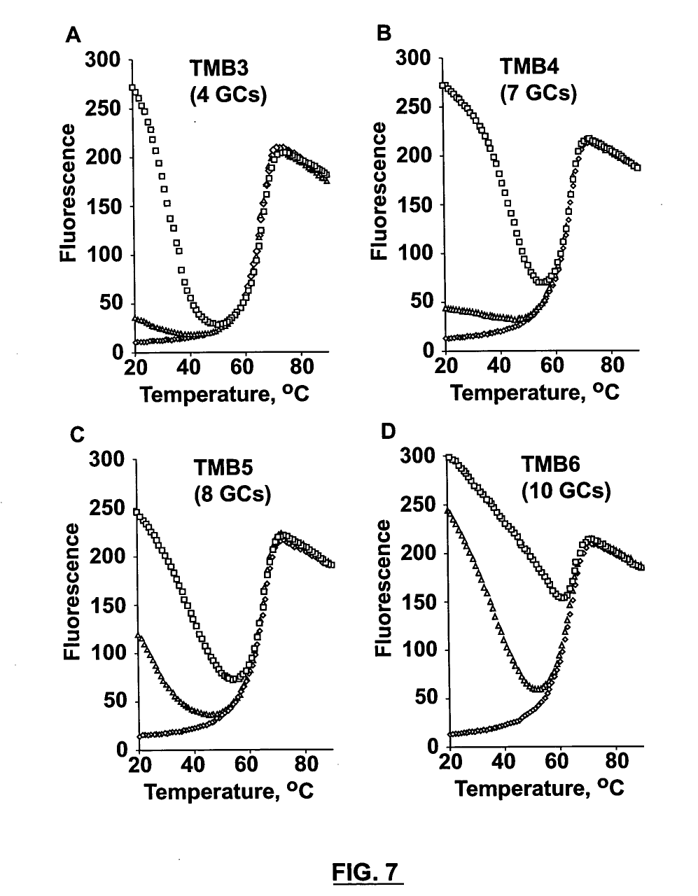

To demonstrate the general utility of a single set of FDNA and QDNA for multiple molecular beacon assembling, additional TMBs were constructed using different LDNA molecules and the common FDNA1 and QDNA1 pair were prepared. The results are shown in FIG. 7. Each LDNA was designed to form a hairpin structure with the universal set of stem-1, stem-2 and stem-3 but with a unique 15-nt loop sequence for target binding. FIGS. 7A, 7B, 7C and 7D illustrate the thermal denaturation profiles of the four TMBs obtained under three conditions: in the absence of target (diamonds), in the presence of a match target (squares), and in the mixture containing a mismatch target (triangles). As expected, all three new tripartite molecular beacons can signal the presence of match nucleic acid targets by large fluorescence intensity change. They also exhibited an ability to discriminate against single-mismatch targets. Consistent with the findings previously reported for standard molecular beacons (11), it was found that the temperature adequate for carrying out single-mismatch discrimination was dependent on the GC content of the target sequence. When the target is AT-rich (as in TMB3 and TMB4), the tripartite molecular beacons demonstrate a high level of performance in discrimination in low temperature range. When the GC content is sufficiently high (as in TMB5 and TMB6), the single-mismatch discrimination can be achieved in high temperature range. For example, the optimal temperature for TMB6 (its target is GC-rich with 67% GC content) was near 50° C., while TMB3, whose target is AT-rich with 73% ATM content, exhibited a large fold of discrimination even at 20° C.

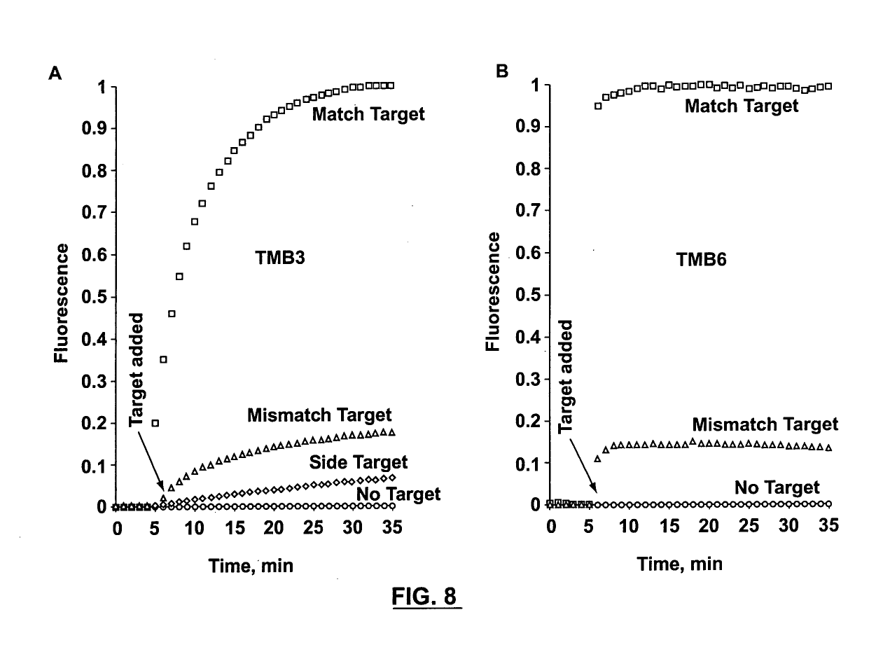

TMB3 (the AT-rich sequence) and TMB6 (the GC-rich sequence) were examined for the real-time signaling capability at a chosen temperature suitable for single mismatch discrimination (22° C. for TMB3 and 50° C. for TMB6) and the results are shown in FIGS. 8A and 8B, respectively. Fluorescence intensities were normalized and a “side target” was also used for TMB3. In an exemplary experiment, solutions containing each TMB were incubated at the designated temperature—first for 5 minutes in the absence of any target, followed by the addition of water (i.e., no target; circles), the mismatch target (triangles) or the match target (squares), and the resultant mixtures were further incubated for 30 more minutes. The fluorescence intensity of each solution was monitored continuously before and after the target introduction. The results indicate that TMBs can be used to effectively discriminate against targets differing by a single nucleotide for both AT-rich and GC-rich targets. The performance levels of the TMBs were similar to those described previously for standard molecular beacons with similar target sequences (1, 4, 11).

Although a TMB is intended for the detection of a DNA target that can form specific Watson-Crick base pairs with the loop sequence of the LDNA (see FIG. 2), it is possible that undesirable interactions that disrupt the formation of stem-1 can lead to false positive results. One possible scenario is that a DNA target might give rise to a false positive signal by binding to the LDNA segment consisting of one of the two complementary sequences of the stem-1 and its nearby nucleotides on each side. To assess the level of interference that might occur in this particular scenario, a special DNA target, ST-1 (ST stands for “side target”), was used to test the false signaling possibility with TMB3. ST-1 contained a 15-nt sequence (the same length as the loop-binding sequences used as the targets throughout this study) intended to disrupt the stem-1 of TMB3 by forming Watson-Crick base pairs with the first seven nucleotides of the stem-1 (as the 5′ complementary sequence of the stem-1) as well as the 8 nearby nucleotides (4 on each side of the stem-1). Only a very weak signal was produced with the introduction of ST-1 (FIG. 5A, the data series in diamonds) and the fluorescence intensity was even lower than that seen with the mismatch target. Therefore, the interference caused by the hypothetical stem disruption is not significant.

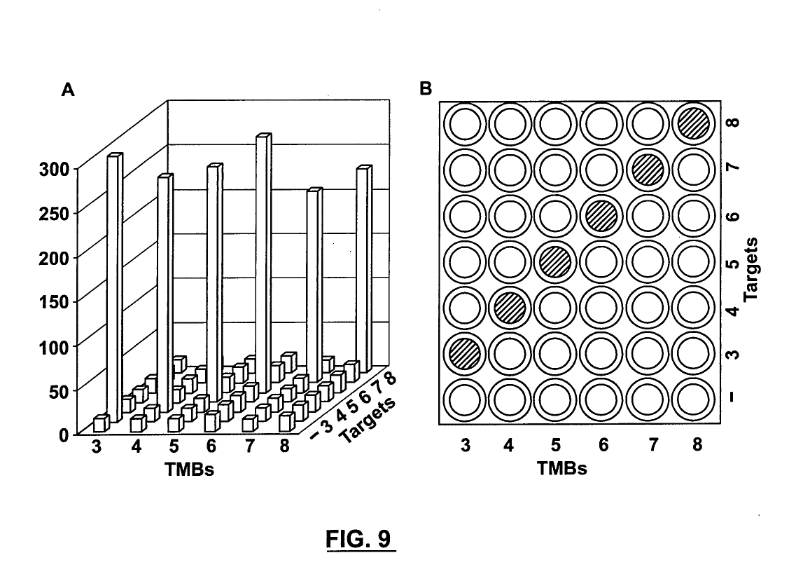

To further demonstrate the general utility of common FDNA/QDNA pair, a simple array experiment for target sensing by fluorescence was conducted. In addition to TMB3-6, two new TMBs, TMB7 and TMB8, that again contained the common set of stem-1, stem-2 and stem-3 but different probing sequences were included for the experiment. Fluorescence intensity of each tripartite molecular beacon in the presence of each DNA target determined at 22° C. is plotted in FIG. 9A. The results indicate that each tripartite molecular beacon emits a very strong fluorescence in the presence of the match target and but exhibits very low background fluorescence in the presence of each of the unintended targets. For instance, TMB7 had a fluorescence intensity of 217 in the presence of T7, but only had fluorescence readings between 14-15 when the other five nondesirable targets were used (the background fluorescence at 13.5). The solutions used for FIG. 9A were also placed in microplate wells and scanned for obtaining a fluorimage. The results are shown in FIG. 9B. Only samples that contained the match target were able to give rise to detectable fluorescent signals. Each TMB was also examined for match target detection in the presence of all six targets (six-target mixture) as well as in the presence of only five unintended targets (five-target mixture) and was found to fluoresce at its maximal capability in the six-target mixture and only emit fluorescence at the background level in the five-target mixture (data not shown). These data clearly indicate the general applicability of FDNA and QDNA as universal probes in setting up parallel molecular beacons for high throughput applications.

The results indicated that the tripartite molecular beacons of the present invention have a high performance level and are practical to use. In summary, in the absence of a nucleic acid target, a tripartite molecular beacon forms a closed structure with three stems and a loop. In this structure, the fluorophore is situated in short distance to the quencher and only low background fluorescence can be observed. When the perfectly matched target nucleic acid is introduced into the solution, a TMB undergoes a structural transformation from the closed and non-fluorescent state to the open and signaling state, reporting the presence of its complementary target. Fluorescence signaling by a TMB is highly specific and a single base mutation within the probe sequence usually results in very significant signal reduction. For single nucleotide discrimination, tripartite molecular beacons also have a capability similar to standard molecular beacons. This was perfectly illustrated by the comparison of MB1 and TMB1. From 20° C. to 40° C., MB1 has a match/mismatch fluorescence ratio between 7 to 9.5 while the ratio for TMB1 holds steady at 7.5. From the comparison of MB1 and TMB1 (FIG. 4) and as well as from the comparison of several other MBs and related TMBs, it is clearly apparent that related MBs and TMBs have very comparable abilities in accurately reporting the presence of match nucleic acid targets and in discriminating targets differing only in single point mutations or single base deletions.

The hairpin structures of TMBs appear to have somewhat decreased melting points as compared to related MBs with identical internal stem-loop sequences. This is likely caused by the difficulty of TMBs in forming the outside edge base pair in Stem 1. This factor needs to be considered when designing TMBs with a desired melting point. The melting points of TMB can still be accurately predicted using M-fold program if the base-pair at the outside edge of Stem 1 is ignored. A convenient way to do this is to first design a stem-loop structure with desired melting point and then to add a “fake” base-pair to the outside edge of the Stem 1. The two bases in this dummy “base-pair” will of course not associate (or not fully associate) when the TMB is fully assembled, therefore their addition will not significantly affect the desired melting point.

Compared to prior molecular beacons, tripartite molecular beacons have the significant advantage that they can be easily adapted for high throughput applications that demand a great number of probes. With a single set of F-DNA and Q-DNA and a series of standard oligonucleotides as L DNAs, a variety of tripartite beacons can easily be assembled for detecting different nucleic acids. The use of tripartite molecular beacons is not only more cost-effective than the use of standard molecular beacons, but also eliminates the tedious procedures involved in synthesizing and purifying each double-labeled DNA probe.

Tripartite molecular beacons also have the advantage of greater flexibility in the choice of fluorophores that can be used. For example, a large number of nucleic acid samples can be probed with two or more fluorophores using the tripartitie molecular beacon approach without the significant increase in cost that would be associated with a standard molecular beacon approach. This is because same L-DNAs and the same Q-DNA can always be used and the additional cost to make new F-DNAs labeled with different fluorophores is fairly small.

Tripartite molecular beacons are also well suited for the construction of wavelength-shifting molecular beacons. A wavelength-shifting molecular beacon uses three labels: a quencher at 3′ end and two fluorophores (harvester fluorophore and emitter fluorophore) located in short distance at the 5′ end (Tyagi et al., 2000). The harvester fluorophore is chosen so that it efficiently absorbs energy from the available monochromatic light source and the absorbed energy is not emitted as fluorescence but transferred to the quencher (in the closed state) or to the emitter fluorophore. It has been found that wavelength-shifting molecular beacons are substantially brighter than conventional molecular beacons that contain a fluorophore that cannot efficiently absorb energy from the available monochromatic light source. Therefore, wavelength-shifting molecular beacons can significantly improve and simplify multiplex detections.

The tripartite molecular beacons of the present invention are also useful in the preparation of molecular beacon microarrays. In the past several years, DNA microarray technology has attracted tremendous interests among biologists (Ramsay, 1998; Whitecombe et al., 1998; Burns, M. A. et al., 1998; Case-Green et al., 1998) because this new platform technology allows massively parallel gene expression and gene discovery studies. DNA microarrays are arrays of oligonucleotide probes produced by either masking techniques or liquid dispersing methods (Chee, M. et al., 1996; Schena et al., 1995; McGall, et al., 1996). Although this technology is in commercial use and has yielded vast amounts of genetic and cellular information, all current DNA array approaches require the labeling of nucleic acid targets with various fluorophores. Target labeling is not only time-consuming but it can change the levels of targets originally present in a sample. With the use of molecular beacons, there is no need to label nucleic acid targets. However, because of the need to covalently attach a fluorophore and a quencher to each sequence and the associated extremely high cost, the use of standard unimolecular beacons in DNA microarrays is not practical. This problem is addressed by the use of the tripartite molecular beacons of the present invention.

Since only unmodified oligo-deoxyribo-nucleotides of tripartite molecular beacons need to be immobilized on the array surface, methods that are currently in use for coating microarrays with synthetic DNA oligo-deoxyribo-nucleotides can be directly used to immobilize LDNAs. FDNA and QDNA can then be supplied as a universal stock solution that can be simply mixed with the sample of interest during the hybridization step. Fluorescence is generated during the hybridization and thus, there is no need to label nucleic acid targets.

The tripartite molecular beacons of the present invention are particularly suited for making molecular beacon arrays. Since only normal oligonucleotides (L-DNAs) need to be immobilized on the array surface, methods that are currently under use for coating microarrays with synthetic DNA oligonucleotides can be used to coat with L-DNAs. F-DNA and Q-DNA can then be supplied as a universal stock solution that can be directly mixed with sample of interest during hybridization. Fluorescence is generated during the hybridization and thus, there is no need to label nucleic acid targets. Thus, the present invention also provides kits for the generation of tripartite molecular beacons.

Tripartite molecular beacons have a high performance similar to the standard molecular beacons and fluorescence signaling by tripartite molecular beacons is highly specific. A single base mutation within the target sequence generates a significant signal reduction.

Since only unmodified oligo-deoxyribo-nucleotides of tripartite molecular beacons need to be immobilized on the array surface, standard techniques for coating microarrays with synthetic DNA oligo-deoxyribo-nucleotides can be used to immobilize L-DNAs. F-DNA and Q-DNA can then be supplied as a universal stock solution that can be simply mixed with the sample of interest during the hybridization step. Fluorescence is generated during the hybridization and thus, there is no need to label nucleic acid targets.

The present invention also provides kits for the construction of tripartite molecular beacons. The kit typically includes an L-DNA which may include a particular probe sequence or a multiple cloning site where one can insert a probe sequence of interest. The kit also includes a F-DNA and a Q-DNA for hybridization to the L-DNA.

The present invention provides tripartite molecular beacons which are as effective as standard molecular beacons in signaling the presence of matching nucleic acid targets and in precisely discriminating targets that differ by a single nucleotide. Due to the nature of the tripartite molecular beacon, the L-DNA provides the capability for surface immobilization through free DNA.

A single set of FDNA and QDNA can be used to construct multiple TMBs for detecting matching targets without false signaling. With the increased assembling flexibility, tripartite molecular beacons are more cost-effective for applications that demand a large number of DNA probes and more compatible with surface immobilization

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

Methods of synthetic chemistry, protein and peptide chemistry and molecular biology, referred to but not explicitly described in this disclosure and examples are reported in the scientific literature and are well known to those skilled in the art.

### Example 1

### Oligonucleotides

Normal and modified oligonucleotides were all prepared by automated DNA synthesis using standard cyanoethylphosphoramidite chemistry (Keck Biotechnology Resource Laboratory, Yale University; Central Facility, McMaster University). Molecular beacons used for our studies contained fluorescein as the fluorophore and/or 4-(4-dimethylaminophenylazo)benzoic acid (DABCYL) as the quencher. Fluorescein and DABCYL were placed on the 5′ and 3′ ends of relevant oligonucleotides, respectively. 5′-fluorescein and 3′-DABCYL DNAs were synthesized by automated DNA synthesis with the use of 5′-fluorescein phosphoramidite and 3′-DABCYL-derivatized controlled pore glass (CPG) (Glen Research, Sterling, Va.).

Unmodified DNA oligonucleotides were purified by 10% preparative denaturing ( 8 M urea) polyacrylamide gel electrophoresis (PAGE), followed by elution and ethanol precipitation. 5′-fluorescein and/or 3′-DABCYL modified oligonucleotides were purified by reverse phase high-pressure liquid chromatography (RP-HPLC). HPLC separation was performed on a Beckman-Coulter HPLC System Gold with 168 Diode Array detector. HPLC column was 1 mm×2 mm C8 column. Two buffer systems were used with Buffer A being 0.1 M triethylammonium acetate (TEAR, pH 6.5) and Buffer B being 100% acetonitrile (All chemical reagents were purchased from Sigma). The best separation results can be achieved by a non-linear elution gradient (10% B for 10 min, 10% B to 40% B in 65 min) at a flow rate of 1 ml/min. The main peak was found to have very strong absorption at both 260 nm and 491 nm. The DNA within ⅔ peak-width was collected and dried under vacuum.

Purified oligonucleotides were dissolved in water and their concentrations were determined spectroscopically. All chemical reagents were purchased from Sigma.

### Example 2

### Fluorescence Measurements

The following concentrations were used for various oligonucleotides (if not otherwise specified): 100 nM for fluorophores, 200 nM for hairpin DNA, 300 nM for quenchers and 600 nM for complementary DNA target. All measurements were made in 1500-p1 solutions containing 500 mM NaCl, 3.5 MM MgCl2 and 10 mM Tris-HCl (pH 8.3). The fluorescence of molecular beacon mixtures was measured on a Cary Eclipse Fluorescence Spectrophotometer (Varian) and with excitation at 490 nm and emission at 520 nm.

For obtaining the thermal denaturation profile of a particular reaction mixture, the DNA solution was heated to 90° C. for 5 min, and the temperature was then decreased from 90° C. to 20° C. at a rate of 1° C./min. A reading was made automatically for every 0.5° C. decrease.

### References

Bonnet, G., Krichevsky, O., and Libchaber, A. Kinetics of conformational fluctuations in DNA hairpin-loops PNAS 1998 95: 8602-8606. 5 Bonnet G, Tyagi S, Libchaber A, and Kramer F R (1999) Thermodynamic basis of the enhanced specificity of structured DNA probes. Proc Natl Acad Sci USA 96, 6171-6176.

Burns, M. A. et al. An integrated nanoliter DNA analysis device. Science 282:484-487 (1998).

Case-Green, S. C., Mir, K. U., Pritchard, C. E. & Southern, E. M. Analyzing genetic information with DNA arrays. Curr. Opin. Chem. Biol. 2:404-410 (1998).

Chee, M. et al. Accessing genetic information with high-density DNA arrays. Science274: 610-614 (1996).

Giesendorf, B. A. J. et al. Molecular beacons: a new approach for semiautomated mutation analysis. Clin. Chem. 44, 482-486 (1998).

Kostrikis, L. G., Tyagi, S., Mhlanga, M. M., Ho, D. D. & Kramer, F. R. Spectral genotyping of human alleles. Science 279, 1228-1229 (1998). 25 Kostrikis, L. G. et al. A chemokine receptor CCR2 allele delays HIV-1 disease progression and is associated with a CCR5 promoter mutation. Nat. Med. 4, 350-353 (1998).

Leone, G., van Schijndel, H., van Gemen, B., Kramer, F. R. & Schoen, C. D Molecular beacon probes combined with amplification by NASBA enable homogeneous, real-time detection of RNA. Nucleic Acids Res. 26, 2150-2155 (1998).

Marras, S. A. E., Kramer, F. R. & Tyagi, S. Multiplex detection of single nucleotide variations using molecular beacons. Genet. Anal. 14, 151-156 (1999).

Matsuo, T: In situ visualization of messenger RNA for basic fibroblast growth factor in living cells. Biochim. Biophys. Actal 379, 178-184 (1998).

McGall, G., Labadie, J., Brock, P., Wallraff, G., Nguyen, T. & Hinsberg, W. Light directed synthesis of high-density oligonucleotide arrays using semiconductor pho-toresists. Proc. Natl. Acad. Sci. USA 93:13555-13560 (1996).

Piatek, A. S. et al. Molecular beacon sequence analysis for detecting drug resistance in Mycobacterium tuberculosis. Nat. Biotechnol. 16, 359-363 1998).

Ramsay, G. DNA chips: state-of-the-art. Nat. Biotechnol. 16:40-44 (1998).

Schena, M., Shalon, D., Davis, R. W. & Brown, P. O. Quantitative monitoring of gene expression patterns with a complementary DNA microarray. Science 270: 467-470 1995). 25 Sokol, D. L., Zhang, X., Lu, P. & Gewirtz, A. M. Real time detection of DNA-RNA hybridization in living cells. Proc. Natl. Acad. Sci. USA 95, 1153811543 (1998).

Tyagi, S., Bratu, D. P. & Kramer, F. R. Multicolor molecular beacons for allele dis-crimination. Nat. Biotechnol. 16, 49-53 (1998).

Tyagi, S. & Kramer, F. R. Molecular beacons: probes that fluoresce upon hybridization. Nat. Biotechnol. 14, 303-308 (1996).

Tyagi, S, Marras, S. A. E., and Kramer, F. R. (2000). Wavelength-shifting molecular beacons. Nat Biotechnol 18, 1191-1196.

Whitecombe, D., Newton, C. R. & Little, S. Advantages in approaches to DNAbased diagnostics. Curr. Opin. Biotechnol. 9:602-608 (1998). 10 Vet, J. A. M. et al. Multiplex detection of four pathogenic retroviruses using mole-cular beacons. Proc. Natl. Acad. Sci. USA 96, 6394-6399 (1999).

I claim:

1. A tripartite probe comprising:
a)a first oligonucleotide having a first end segment, a second end segment and a probe segment intermediate said first and second end segments;
b)a second, fluorescent-labeled oligonucleotide (F-DNA) hybridized to said first end segment; and
c)a third, quencher-modified oligonucleotide (Q-DNA) hybridized to said second end segment,
wherein said first end segment and said second end segment have complementary regions capable of forming the first oligonucleotide into a stem-loop structure.

2. A probe according to claim 1, wherein the first end segment comprises a first oligonucleotide-binding segment and a first complementarity segment adjacent to said first oligonucleotide-binding segment, and the second end segment comprises a second complementarity segment complementary to said first complementarity segment and a second oligonucleotide-binding segment adjacent to said second complementarity segment and wherein said F-DNA hybridizes to said first oligonucleotide-binding segment and said Q-DNA hybridizes to said second oligonucleotide-binding segment.

3. A probe according to claim 1, wherein said probe segment comprises a sequence complementary to a target sequence.

4. The probe of claim 3, wherein, in the absence of a target sequence, said first complementarity segment and said second complementarity sequence hybridize to form a duplex, thereby bringing the F-DNA and the Q-DNA into proximity whereby fluorescence from the F-DNA is quenched by the Q-DNA.

5. The probe of claim 3, wherein, in the presence of a target sequence, said probe segment binds to said target sequence and forms a probe-target duplex, thereby spatially separating the F-DNA and the Q-DNA whereby fluorescence from the F-DNA can be detected.

6. The probe of claim 5, wherein the melting point of the probe-target duplex is higher than the melting point of the stem formed between the complementarity regions.

7. The probe of claim 1, wherein said fluorophore is covalently linked to one end of said second oligonucleotide.

8. The probe of claim 1, wherein said second oligonucleotide comprises at least one fluorescent nucleotide analog.

9. The probe of claim 1, wherein said third oligonucleotide has a quencher moiety attached at one end.

10. The probe of claim 1, wherein said third oligonucleotide incorporates quenching nucleotides.

11. A kit for the detection of a target sequence, said kit comprising:
i) a loop oligonucleotide (L-DNA) comprising a probe sequence and complementary sequences on each side of said probe sequence;
ii) a fluorescent labeled oligonucleotide capable of hybridizing to said loop oligonucleotide on one side of said probe sequence; and
iii) a quencher modified oligonucleotide capable of hybridizing to the loop oligonucleotide on the other side of the probe sequence.

12. A kit according to claim 11, wherein said probe sequence comprises a sequence complementary to a target sequence.

13. A kit according to claim 11, wherein said probe sequence comprises a restriction enzyme cloning site.

14. A method of preparing an array for detection of nucleic acid sequences comprising the steps of:
i) providing a loop oligonucleotide having a probe sequence and complementary end segments capable of forming a stem-loop structure;
ii) immobilizing said loop oligonucleotide on a surface;
iii) incubating said surface with a fluorophore labeled oligonucleotide complementary to a first region of said loop oligonucleotide and a quencher modified oligonucleotide complementary to a second region o f said loop oligonucleotide wherein said fluorophore labeled oligonucleotide and said quencher modified oligonucleotide hybridize to said loop oligonucleotide and fluorescence is detected when said probe sequence binds to a complementary target sequence.

15. A method according to claim 14, wherein said loop oligonucleotide is immobilized on the surface through free DNA ends.

16. A method according to claim 14, wherein said loop oligonucleotide, said fluorophore labeled oligonucleotide and said quencher modified oligonucleotide are combined prior to immobilization on the surface.

17. A method according to claim 14, wherein the fluorophore labeled oligonucleotide and the quencher-modified oligonucleotide are added after the loop oligonucleotide is immobilized.

18. A method according to claim 14 wherein said fluorophore labeled oligonucleotide and said quencher modified oligonucleotide are added sequentially.


SEQUENCE LISTING (15 sequences)

SEQ ID NO: 1 - DNA, 57 bp, Artificial Sequence
  Misc_feature (1)..(57): Oligonucleotides 1-57 were prepared by automated synthesis
cctgccacgctccgcgcgagccaccaaatatgatatgctcgcctcgcaccgtccacc

SEQ ID NO: 2 - DNA, 27 bp, Artificial Sequence
  Misc_feature (1)..(27): Oligonucleotides 1-27 were prepared by automated synthesis
gcgagccaccaaatatgatatgctcgc

SEQ ID NO: 3 - DNA, 30 bp, Artificial Sequence
  Misc_feature (1)..(30): Oligonucleotides 1-30 were prepared by automated synthesis
tactcttatatcatatttggtgtttgcttt

SEQ ID NO: 4 - DNA, 30 bp, Artificial Sequence
  Misc_feature (1)..(30): Oligonucleotides 1-30 were prepared by automated synthesis
tactcttatatcatctttggtgtttgcttt

SEQ ID NO: 5 - DNA, 57 bp, Artificial Sequence; identical to SEQ ID NO: 1
  Misc_feature (1)..(57): Oligonucleotides 1-57 were prepared by automated synthesis

SEQ ID NO: 6 - DNA, 27 bp, Artificial Sequence
  Misc_feature (1)..(27): Oligonucleotides 1-27 were prepared by automated synthesis
gcgagccaccaaatatgatatgctcgc

SEQ ID NO: 7 - DNA, 59 bp, Artificial Sequence
  Misc_feature (1)..(59): Oligonucleotides 1-59 were prepared by automated synthesis
cctgccacgctccgcagcgagccaccaaatatgatatgctcgctctcgcaccgtccacc

SEQ ID NO: 8 - DNA, 59 bp, Artificial Sequence
  Misc_feature (1)..(59): Oligonucleotides 1-59 were prepared by automated synthesis
cctgccacgctccgcggcgagccaccaaatatgatatgctcgccctcgcaccgtccacc

SEQ ID NO: 9 - DNA, 15 bp, Artificial Sequence
  Misc_feature (1)..(15): Oligonucleotides 1-15 were prepared by automated synthesis
gcggagcgtggcagg

SEQ ID NO: 10 - DNA, 15 bp, Artificial Sequence
  Misc_feature (1)..(15): Oligonucleotides 1-15 were prepared by automated synthesis
cctgccacgctccgc

SEQ ID NO: 11 - DNA, 15 bp, Artificial Sequence
  Misc_feature (1)..(15): Oligonucleotides 1-15 were prepared by automated synthesis
ggtggacggtgcgag

SEQ ID NO: 12 - DNA, 15 bp, Artificial Sequence
  Misc_feature (1)..(15): Oligonucleotides 1-15 were prepared by automated synthesis
ctcgcaccgtccacc

SEQ ID NO: 13 - DNA, 15 bp, Artificial Sequence
  Misc_feature (1)..(15): Oligonucleotides 1-15 were prepared by automated synthesis
ggtggacggtgcgag

SEQ ID NO: 14 - DNA, 15 bp, Artificial Sequence
  Misc_feature (1)..(15): Oligonucleotides 1-15 were prepared by automated synthesis
gcggagcgtggcagg

SEQ ID NO: 15 - DNA, 31 bp, Artificial Sequence
  Misc_feature (1)..(31): Oligonucleotides 1-31 were prepared by automated synthesis
cctgccacgctccgctctcgcaccgtccacc